(19) United States
(12) Patent Application Publication
(10) Pub. No.: US 2007/0060997 A1
(43) Pub. Date: Mar. 15, 2007

(54) Multi-lumen steerable catheter

(76) Inventor: Jan de Boer, De Wilgen (NL)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/227,788

(22) Filed: Sep. 15, 2005

(51) Int. Cl. A61F 2/06

(52) U.S. Cl. 623/1.11; 604/95.04

(57) ABSTRACT

A steerable catheter may have a flexible shaft extending between a proximal and distal end, with a hub affixed to the proximal end, and at least one actuator for causing the catheter distal end to move or bend in a desired direction. The catheter shaft defines at least one passage or steering lumen, at least one of which has a closed distal end. The hub may have a number of actuators, such that each actuator may affect the pressure of a pressure medium in a steering lumen. When specific actuators are activated, the pressures in each of the steering lumens may be disparately raised and/or lowered, so as to cause a portion of the catheter shaft to tend to bend in a specific direction. For example, one or some of the actuators may be used to change the pressure of the associated steering lumens, causing the catheter shaft to bend. Also, in the case of a steerable catheter having more than one actuator, all of the actuators may be activated to raise the pressure of all of the steering lumens, tending to temporarily increase the column stiffness and pushability of the catheter shaft.

### BACKGROUND AND SUMMARY OF THE INVENTION

1. Technical Background

The present invention relates generally to medical devices, and more particularly to a device for facilitating the loading of a guidewire into a lumen defined by a catheter.

2. Discussion

There are many different kinds and types of catheters, including for example balloon catheters, diagnostic catheters, guiding catheters, stent delivery system catheters, injection catheters, gene therapy catheters, electrophysiology catheters, therapeutic drug delivery catheters, ultrasound catheters, laser angioplasty catheters, etc.

Catheters are often intended to follow a specific path through body passages selected by a physician. Accordingly, it may be desirable to provide a catheter with a shaft capable of bending when a physician wishes. Referred to as a “steerable” catheter, this type of catheter allows the physician to more readily advance it along a desired path. For example, such a bending or steerable feature assists the physician to select one of two or more branching paths.

Structurally, catheters may have a flexible shaft extending between a proximal end and a distal end, and may define one or more tubular passages or “lumens” extending through part or all of the catheter shaft. Such lumens often have one or more openings, referred to as “ports,” or a lumen may have a closed lumen.

When a lumen is adapted to slidingly receive a guidewire, it is referred to as a “guidewire lumen,” and it will generally have a proximal and distal “guidewire port.” The distal guidewire port is often at or near the catheter shaft distal end.

A hub is often affixed to the catheter shaft proximal end. The hub may serve a variety of functions, including providing a handle for manipulating the catheter, and/or defining proximal port(s) communicating with lumen(s) defined by the catheter shaft. When the catheter has a guidewire lumen, a proximal guidewire port may be located at some point along the sidewall of the catheter shaft, or a hub may define the proximal guidewire port.

A guidewire has a flexible wire-like structure extending from a proximal end to a distal end. The guidewire will usually be of a size selected to fit into and slide within a corresponding guidewire lumen of a catheter.

Examples of steerable catheters are described in U.S. Pat. No. 6,183,435 to Bumbalough et al., entitled “Multi-Directional Steerable Catheters and Control Handles,” which is commonly owned with the present application. One example design shown in this patent uses “puller wires”, which extend from a handle to distal ends anchored in the tip section of the catheter.

In contrast, a steerable catheter may be provided with one or more “steering lumens” defined inside the shaft sidewall, each of which follows a path that is away from the shaft centerline. Each steering lumen contains a pressurizable fluid, so that when the fluid pressure in a steering lumen is changed, a portion of the catheter shaft will tend to bend in a specific direction. Likewise, if the catheter includes multiple steering lumens, and the fluid pressure in the steering lumens is disparately raised and/or lowered, the catheter shaft will tend to bend.

Also, in the case of a steerable catheter having more than one steering lumen, if the fluid pressure in all of the lumens is raised, the column stiffness and pushability of the catheter shaft may be temporarily increased.

The terms “tube” and “tubular” are used in their broadest sense, to encompass any structure arranged at a radial distance around a longitudinal axis. Accordingly, the terms “tube” and “tubular” include any structure that (i) is cylindrical or not, such as for example an elliptical or polygonal cross-section, or any other regular or irregular cross-section; (ii) has a different or changing cross-section along its length; (iii) is arranged around a straight, curving, bent or discontinuous longitudinal axis; (iv) has an imperforate surface, or a periodic or other perforate, irregular or gapped surface or cross-section; (v) is spaced uniformly or irregularly, including being spaced varying radial distances from the longitudinal axis; or (vi) has any desired combination of length or cross-sectional size.

Any suitable material may be used to make catheters and hubs as described, including polymers and other materials suitable for use with medical devices.

Also, designers of steerable catheters according to the present invention may select to include one or more of any feature or element. Accordingly, whenever the singular or plural form of a word is used in this description, such word shall include the appropriate singular and/or plural forms.

It is of course possible to build various kinds and designs of catheters according to the present invention, by various techniques and of various materials, to obtain the desired features. It should be noted that the present invention also relates to methods for making and using steerable catheters, during or in preparation for medical treatment of a patient.

These and various other objects, advantages and features of the invention will become apparent from the following description and claims, when considered in conjunction with the appended drawings. The invention will be explained in greater detail below with reference to the attached drawings of a number of examples of embodiments of the present invention.

### BRIEF DESCRIPTION OF THE DRAWINGS

FIG. 1 is a perspective view of a steerable catheter;

FIG. 2 is a cross-sectional view of the steerable catheter of FIG. 1, along the line 2-2;

FIG. 3 is a cross-sectional view of a steerable catheter;

FIG. 4 is a perspective view of a steerable stent delivery system catheter, with a guidewire;

FIG. 5 is a perspective view of a steerable balloon catheter, with a guidewire;

FIG. 6 is a perspective view of a steerable catheter having a hub with four actuators, in which all actuators are pressed in the direction of the arrows; and

FIG. 7 is a perspective view of the steerable catheter of FIG. 6, in which one actuator is pressed in the direction of the small arrow, causing the catheter distal end to bend in the direction of the curved arrow.

### DETAILED DESCRIPTION OF THE PREFERRED EMBODIMENTS

The following description of the preferred embodiments of the present invention is merely illustrative in nature, and as such it does not limit in any way the present invention, its application, or uses. Numerous modifications may be made by those skilled in the art without departing from the true spirit and scope of the invention.

The drawings depict steerable catheters along the lines of the present invention. In FIG. 1, a steerable catheter 10 has a flexible shaft 12 extending between proximal and distal ends 14 and 16, a hub 18 affixed to the proximal end 14, and a strain relief 20 positioned at the transition. As shown in FIG. 2, the catheter shaft 12 may define a central lumen 22, and in this particular design, four steering lumens 24. The steering lumens 24 are arranged about the periphery of the shaft 12, and are defined inside the shaft sidewall. Each steering lumen 24 in this design follows a path that is parallel to a centerline of the shaft 12, so they can each be described as being “off center.”

The central lumen 22 extends between a distal port 26 and a proximal port 28 defined by the hub 18. This central lumen 22 may be used for a variety of purposes, such as for example: (i) infusion of liquids, including therapeutic drugs, x-ray contrast medium, etc.; and (ii) slidably receiving a flexible guidewire for guiding the catheter system to a desired site for therapeutic treatment.

Each of the steering lumens 24 are filled with one or more fluids. Such fluid(s) may be pressurizable or not, in other words substantially compressible or incompressible. Also, the fluid(s) may be in whatever state is desirable, such for as example liquid or gas. Of course, various suitable fluids may be used.

In the particular embodiment shown in the drawings, if there is more than one fluid in the steering lumens 24, at least one of the fluids may be pressurizable. To contain the fluid(s), each steering lumen 24 has a closed distal end. A proximal end of each steering lumen 24 extends to a proximal end which communicates with the hub 18.

The hub 18 has four actuators 30, each of which is coupled to one of the steering lumens 24. In the particular design shown in FIG. 1, the actuators 30 are in the form of push-buttons. Of course, any suitable type of actuator may be used, including levers, bellows, bladders, pumps, screw-threaded actuators, pistons, hydraulics, etc.

Regardless of what type of actuator is selected, each actuator affects the pressure of the fluid inside the corresponding steering lumen. Accordingly, when an actuator is actuated, the fluid pressure in the corresponding steering lumen is changed, and the catheter shaft tends to bend. In other words, whenever the fluid pressure in the steering lumens 24 is not symmetrically arranged around the centerline, a bending force is generated.

An example of the present fluid-based steering system is illustrated in somewhat diagrammatic form in FIG. 7. As one of the actuators 30 is depressed in the direction of the small arrow, the fluid pressure in the corresponding steering lumen 24 is changed. This pressure change tends to cause the catheter shaft distal end to bend, as shown by the curved arrow in FIG. 7.

Of course, the actuators may be designed so that actuation either raises or lowers the pressure in the corresponding steering lumen. Regardless how the actuators are arranged, each actuator may be arranged to affect the fluid pressure into the corresponding steering lumen in the same way.

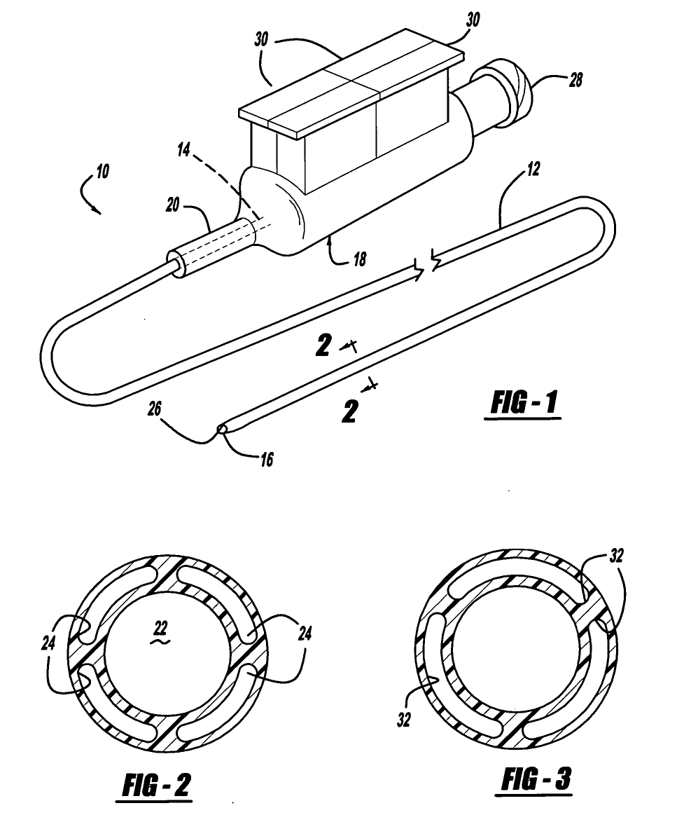

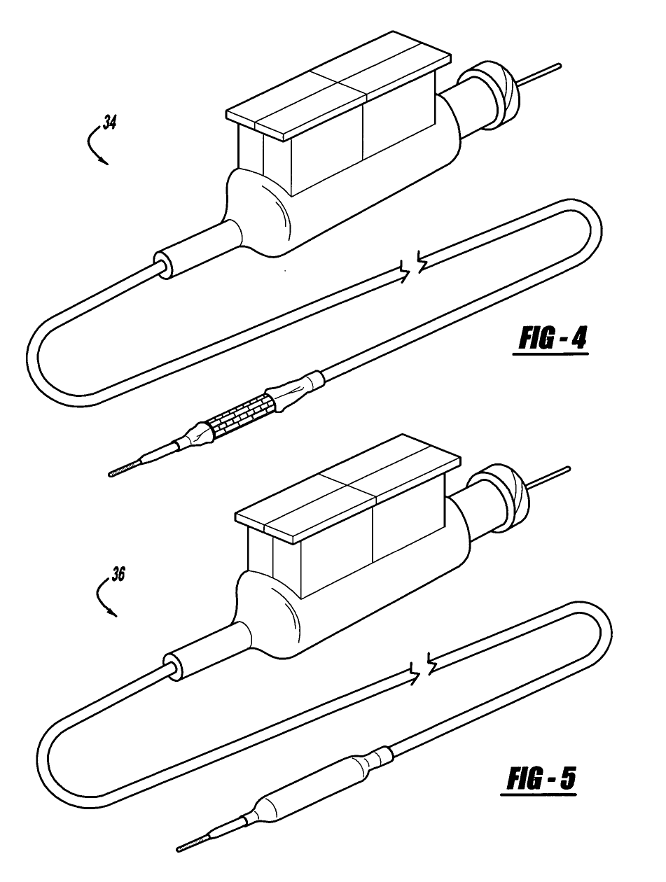

Any suitable number of steering lumens may be selected. For example, FIGS. 2 and 3 show four and three steering lumens 24 and 32, respectively. Regardless, the number of steering lumens equals the number of actuators.

Whatever number of steering lumens and actuators is selected, the catheter shaft will tend to bend whenever the fluid pressures in the steering lumens are not symmetrically distributed around the catheter shaft centerline. In other words, when the steering lumen fluid pressures generate an aggregate off-center pressure, a bending moment will result.

For example, with specific reference to FIG. 3, if the pressure in any one of the three steering lumens 24 is altered to substantially different than the pressures into he other two lumens, a bending force will result. Likewise, substantially changing the pressure in any of the other steering lumens will generate a bending force in another direction.

A slight contrast is a design such as that in FIG. 2 having an even number of steering lumens. If for example opposing pairs of steering lumens have equal fluid pressures (though adjacent steering lumens have different pressures), no bending moment will result. Again, regardless of how many steering lumens there are, an off-center aggregate steering lumen fluid pressure will generate a bending moment.

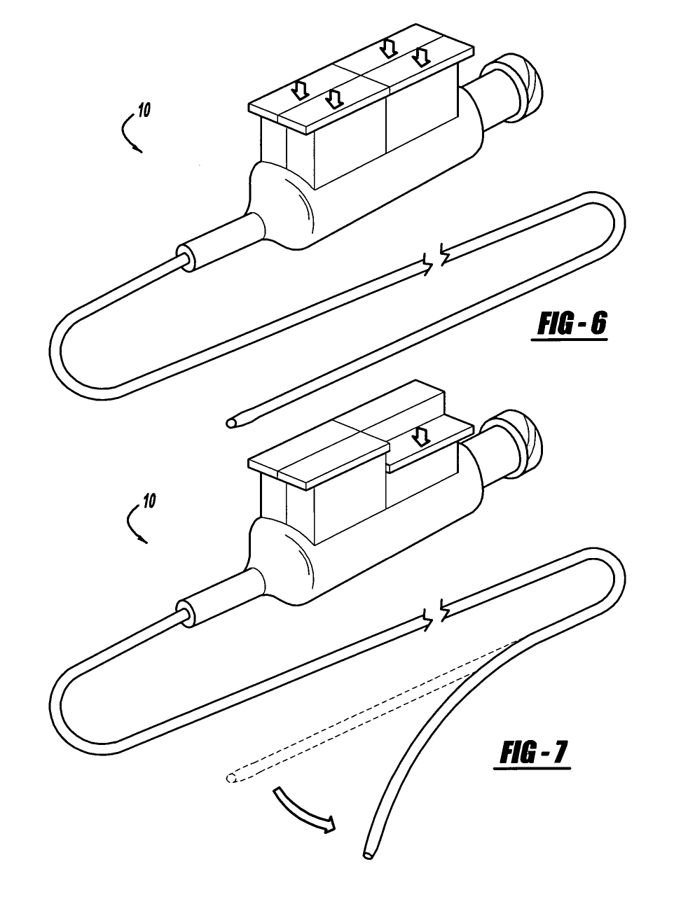

Of course, the fluid-based steering concept of the present invention may be used with most types of catheters having a flexible shaft, including for example the balloon catheter 34 of FIG. 5, and the stent delivery system 36 of FIG. 4.

In addition, another possible feature of the present invention is depicted in FIG. 6, in which all of the actuators are actuated at once. If the actuators are also arranged to increase fluid pressure upon activation, then the fluid pressure will increase in all the steering lumens 24. Accordingly, the physician can selectively alter the stiffness or column strength of the catheter shaft.

Steerable catheters may also be designed for use with a variety of different kinds of catheters. Since there are many different types of catheters which have guidewire lumens and guidewire ports, steerable catheters may be provided for use with any of them. Examples of different types of catheters include balloon catheters, diagnostic catheters, guiding catheters, stent delivery system catheters, injection catheters, gene therapy catheters, electrophysiology catheters, therapeutic drug delivery catheters, ultrasound catheters, laser angioplasty catheters, etc.

Steerable catheters according to the principles of the present invention may be made of any suitable materials, using a variety of methods. Various polymers have the desired characteristics of strength, resilience, flexibility, biocompatibility and endurance. For example, various polymers may be used, such as nylons and other polyamides, as well as polyimides, polycarbonate, polypropelene, ABS, and polyethylenes.

It should be understood that an unlimited number of configurations for the present invention could be realized. The foregoing discussion describes merely exemplary embodiments illustrating the principles of the present invention, the scope of which is recited in the following claims. Those skilled in the art will readily recognize from the description, claims, and drawings that numerous changes and modifications can be made without departing from the spirit and scope of the invention.

What is claimed is:

1. A steerable catheter for therapeutically treating a patient, comprising:
a flexible catheter shaft defining a centerline, and extending between proximal and distal ends; the shaft defining a central lumen extending a proximal and distal port at the proximal and distal ends, and at least one steering lumen extending between positions near the catheter shaft proximal and distal ends;
a hub affixed to the catheter shaft proximal end;
the hub having at least one actuator; wherein the number of steering lumens equals the number of actuators, and each actuator is coupled with a corresponding steering lumen;
wherein each steering lumen follows a path that is off the centerline; a distal end of each steering lumen is closed; and each steering lumen contains a fluid;
such that activation of each actuator causes a corresponding change in pressure of the fluid in the corresponding steering lumen;
such that when the fluid pressure in each steering lumen is not equal, the pressure differential tends to cause the catheter shaft to bend.

2. The steerable catheter of claim 1, wherein activation of an actuator causes an increase in pressure of the fluid in the corresponding steering lumen.

3. The steerable catheter of claim 1, wherein activation of an actuator causes a decrease in pressure of the fluid in the corresponding steering lumen.

4. The steerable catheter of claim 1, wherein the actuators are bi-directional, and are adapted to selectively increase or decrease the pressure of the fluid in the corresponding steering lumen.

5. The steerable catheter of claim 1, wherein the catheter shaft defines three steering lumens.

6. The steerable catheter of claim 1, wherein the catheter shaft defines four steering lumens.

7. The steerable catheter of claim 1, wherein the catheter shaft defines more than one steering lumen and the hub has more than one actuator, and wherein when all the actuators are activated, the pressures of the fluids are increased in all of the lumens, tending to temporarily increase the column stiffness and pushability of the catheter shaft.

8. A steerable balloon catheter for therapeutically treating a patient, comprising:
a flexible catheter shaft defining a centerline, and extending between proximal and distal ends; the shaft defining a central lumen extending a proximal and distal port at the proximal and distal ends, and at least one steering lumen extending between positions near the catheter shaft proximal and distal ends;
a hub affixed to the catheter shaft proximal end;
the hub having at least one actuator; wherein the number of steering lumens equals the number of actuators, and each actuator is coupled with a corresponding steering lumen;
a balloon affixed to the catheter shaft near its distal end; the catheter shaft defining an inflation lumen extending between an interior of the balloon to an inflation port defined by the hub;
wherein each steering lumen follows a path that is off the centerline; a distal end of each steering lumen is closed; and each steering lumen contains a fluid;
such that activation of each actuator causes a corresponding change in pressure of the fluid in the corresponding steering lumen;
such that when the fluid pressure in each steering lumen is not equal, the pressure differential tends to cause the catheter shaft to bend.

9. The steerable balloon catheter of claim 8, wherein the balloon material is substantially inelastic.

10. The steerable balloon catheter of claim 8, wherein the balloon is an angioplasty balloon.

11. The steerable balloon catheter of claim 8, further comprising a stent crimped around the balloon, such that inflation of the balloon will expand and deploy the stent.

12. The steerable balloon catheter of claim 8, wherein activation of an actuator causes an increase in pressure of the fluid in the corresponding steering lumen.

13. The steerable balloon catheter of claim 8, wherein activation of an actuator causes a decrease in pressure of the fluid in the corresponding steering lumen.

14. The steerable balloon catheter of claim 8, wherein the actuators are bidirectional, and are adapted to selectively increase or decrease the pressure of the fluid in the corresponding steering lumen.

15. The steerable balloon catheter of claim 8, wherein the catheter shaft defines three steering lumens.

16. The steerable balloon catheter of claim 8, wherein the catheter shaft defines four steering lumens.

17. The steerable balloon catheter of claim 8, wherein the catheter shaft defines more than one steering lumen and the hub has more than one actuator, and wherein when all the actuators are activated, the pressures of the fluids are increased in all of the lumens, tending to temporarily increase the column stiffness and pushability of the catheter shaft.
